(19) United States
(12) Patent Application Publication
(10) Pub. No.: US 2013/0123537 A1
(43) Pub. Date: May 16, 2013

(54) PROCESS FOR THE PREPARATION OF LACOSAMIDE

(71) Applicants: K a s s Narayan Garimella, Hyderabad (IN); Subba Reddy Danda, Hyderabad (IN); Shankar Reddy Budidet, Hyderabad (IN); Srinivasachary Katuroju, Hyderabad (IN); Gowrisankar Rao Kaki, Hyderabad (IN); Srinivasa Rao Yatcherla, Hyderabad (IN); Islam Aminul, Hyderabad (IN); Sivakumaran Meenakshisunderam, Hyderabad (IN)

(72) Inventors: K a s s Narayan Garimella, Hyderabad (IN); Subba Reddy Danda, Hyderabad (IN); Shankar Reddy Budidet, Hyderabad (IN); Srinivasachary Katuroju, Hyderabad (IN); Gowrisankar Rao Kaki, Hyderabad (IN); Srinivasa Rao Yatcherla, Hyderabad (IN); Islam Aminul, Hyderabad (IN); Sivakumaran Meenakshisunderam, Hyderabad (IN)

(21) Appl. No.: 13/261,518

(22) PCT Filed: May 16, 2011
(86) PCT No.: PCT/IB2011/001035

§ 371 Date: Nov. 15, 2012

(30) Foreign Application Priority Data
May 17, 2010 (IN) 1382/CHE/2010
Sep. 8, 2010 (IN) 2631/CHE/2010

(51) Int. Cl. C07C 231/12

(52) U.S. Cl. CPC C07C 231/12
USPC 564/158

(57) ABSTRACT

The present invention relates to an improved process for the preparation of Lacosamide of Formula (I), comprising: O-methylating a compound of Formula (V) or a compound of Formula (XX) or a compound of Formula XXII; in the presence of a methylating agent and a base to produce Lacosamide of Formula (I).

### FIELD OF THE INVENTION

The present invention relates to an improved process for the preparation of Lacosamide of Formula (I).

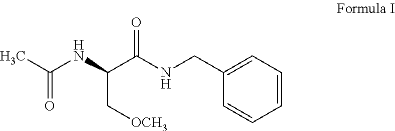 [SMILES: COC[C@@H](NC(C)=O)C(=O)NCc1ccccc1]

### BACKGROUND OF THE INVENTION

Lacosamide is chemically known as (R)-2-acetamido-N-benzyl-3-methoxypropionamide (I).

Lacosamide is an anticonvulsant, which selectively enhances slow inactivation of voltage-gated sodium channels, resulting in stabilization of hyperexitable neuronal membranes and inhibition of repetitive neuronal firing. Lacosamide is marketed under the trade name Vimpat®. It has been approved for the treatment of partial-onset seizures.

Lacosamide and its pharmaceutically acceptable salts are disclosed in U.S. Pat. No. 5,654,301 and RE 38,551 E (U.S. Pat. No. 5,773,475).

According to the process disclosed in US '551, Lacosamide of Formula (I) is prepared by reacting D-Serine (II) with methanol in the presence of HCl to produce D-Serine methyl ester hydrochloride (III), which is reacted with benzylamine to produce (R)—N-benzyl-2-amino-3-hydroxypropionamide (IV), which is further acetylated in the presence of acetic anhydride, followed by recrystallization to produce (R)—N-benzyl-2-acetamido-3-hydroxypropionamide (V). Compound (V) is further reacted with methyl iodide in the presence of silver oxide to produce Lacosamide of Formula (I).

The process is as shown in Scheme-I below:

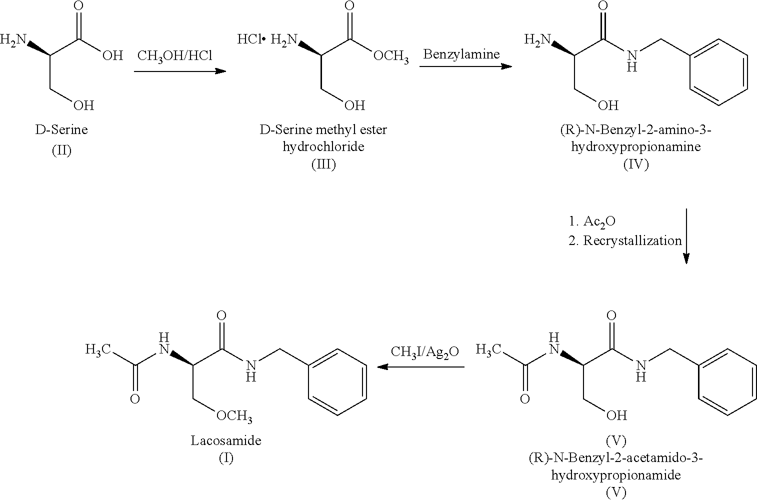 [SMILES: N[C@H](CO)C(=O)O ; COC(=O)[C@H](N)CO ; N[C@H](CO)C(=O)NCc1ccccc1 ; "(II)" ; COC[C@@H](NC(C)=O)C(=O)NCc1ccccc1 ; CC(=O)N[C@H](CO)C(=O)NCc1ccccc1]

US '551 also discloses a variant process for the preparation of Lacosamide of Formula (I), by reacting D-Serine (II) with acetic anhydride in acetic acid to produce (R)-2-acetamido-3-hydroxypropionic acid (VI), which is further reacted with benzylamine to produce (R)—N-benzyl-2-acetamido-3-hydroxypropionamide (V), which is further reacted with methyl iodide in the presence of silver oxide to produce Lacosamide of Formula (I).

The process is as shown in Scheme-II below:

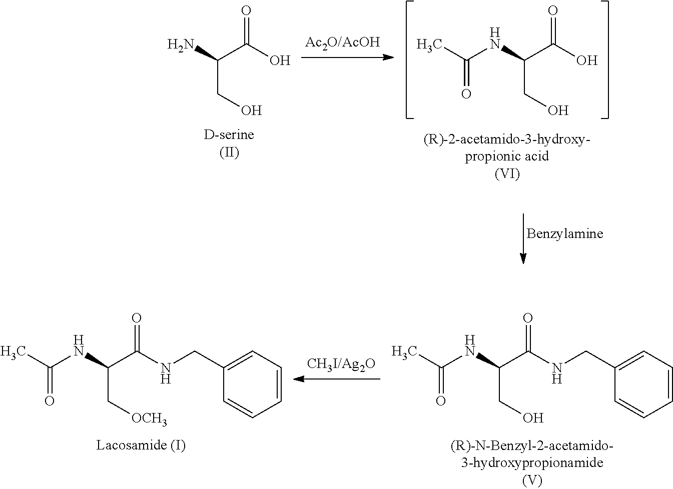

The above processes comprise the benzylamide formation prior to the O-methylation. However, this processes results in Lacosamide with various impurities, which must be removed by column chromatography. Employing column chromatography technique is tedious and laborious and also involves use of large quantities of solvents, and hence is not suitable for industrial scale operations.

US '551 also discloses another variant process for the preparation of Lacosamide of Formula (I), by reacting D-Serine with benzylchloroformate (Cbz-Cl) in the presence of magnesium oxide to produce Cbz-D-Serine (VII), which is further reacted with methyl iodide in the presence of silver oxide and methyl cyanide to produce (R)-methy-2-(carbobenzyloxyamino)-3-methoxypropionate (VIII). Compound (VIII) is treated with anhydrous K₂CO₃ in the presence of aqueous methanol to produce (R)-2-(carbobenzyloxyamino)-3-methoxypropionic acid (IX), which is reacted with benzylamine in the presence of N-methylmorpholine and isobutyl chloroformate in dry THF to produce (R)-2-(carbobenzyloxyamino)-3-methoxypropionamide (X), which is further reacted with hydrogen gas in the presence of palladium-carbon and methanol to produce N-benzyl-2-amino-3-methoxypropionamide (XI).

Acetylation of N-benzyl-2-amino-3-methoxypropionamide (XI) with acetic anhydride in the presence of pyridine in THF to produce Lacosamide of Formula (I).

The process is as shown in Scheme-III below:

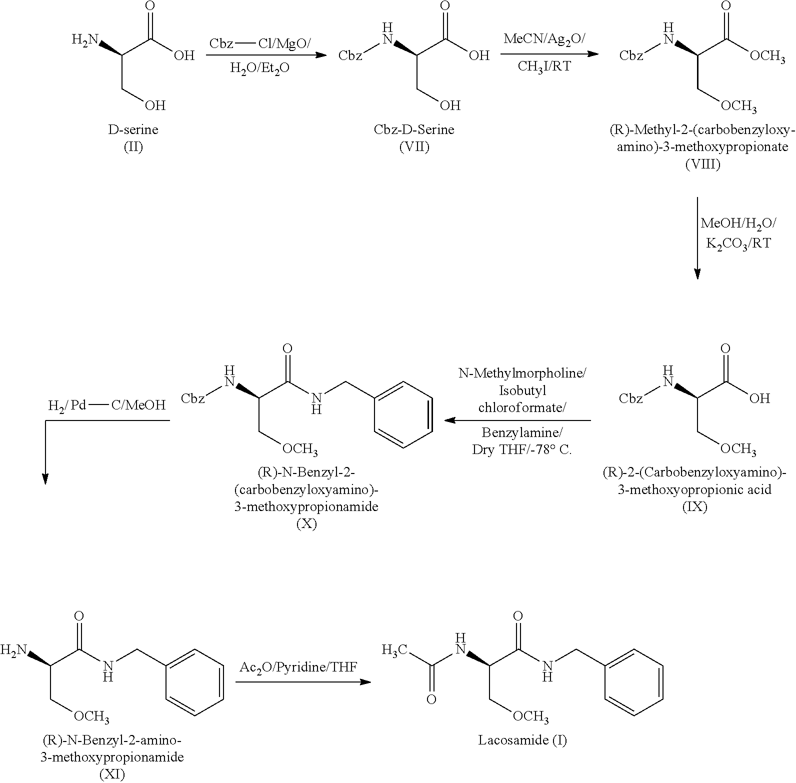

U.S. Pat. No. 6,048,899 discloses another variant process for the preparation of Lacosamide of Formula (I), by reacting D-Serine with benzylchloroformate (Cbz-Cl) in the presence of magnesium oxide to produce Cbz-D-Serine (VII), which is further reacted with benzylamine in the presence of N-methylmorpholine and isobutyl chloroformate to produce (R)—N-benzyl-2-(carbobenzyloxyamino)-3-hydroxypropionamide (Xa), which is reacted with methyl iodide in the presence of silver oxide to produce (R)—N-benzyl-2-(carbobenzyloxyamino)-3-methoxypropionamide (X). Compound (X) which is hydrogenated in the presence of palladium catalyst to produce N-benzyl-2-amino-3-methoxypropionamide (XI). Acetylation of N-benzyl-2-amino-3-methoxy-propionamide (XI) with acetic anhydride in the presence of pyridine in THF to produce Lacosamide of Formula (I).

The process is as shown in Scheme-IV below:

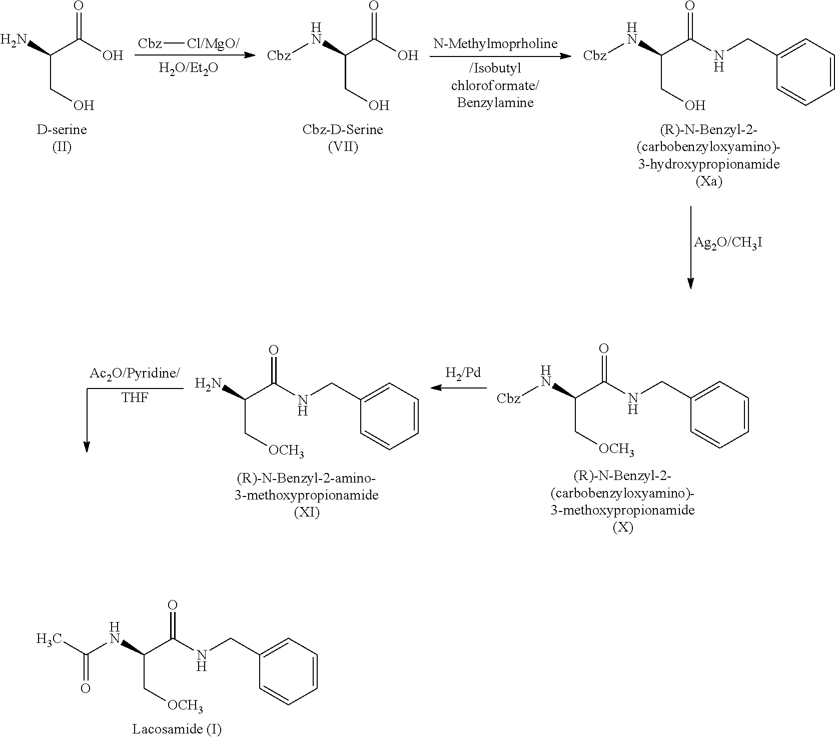

The disadvantage with the above processes is the use of silver oxide in the O-methylation step. This reagent is highly expensive and results in partial racemisation, which reduces the yield. Further, removal of the S-enantiomer of Lacosamide is more difficult at this stage, which requires repeated crystallizations. Additionally, a second and third crystallization reduces yield as some Lacosamide of Formula (I) remains uncrystallized and is not recovered from the liquid phase.

US 2008/0027137 A1 discloses a process for the preparation of Lacosamide of Formula (I), by methylation of N-Boc-D-serine (XII) with dimethyl sulphate and butyllithium or dimethyl sulphate in the presence of a base and phase transfer catalyst (PTC) to produce (R)—N-Boc-amino-3-methoxy-propanoic acid (XIII), which is further reacted with benzylamine in the presence of isobutyl chloroformate and N-methylmorpholine to produce (R)—N-benzyl-2-Boc-amino-3-methoxy-propionamide (XIV). Compound (XIV) is converted to (R)—N-benzyl-2-amino-3-methoxypropionamide (XI) by treating with HCl in water, which is further reacted with acetic anhydride in ethyl acetate to produce Lacosamide of Formula (I).

The process is as shown in Scheme-V below:

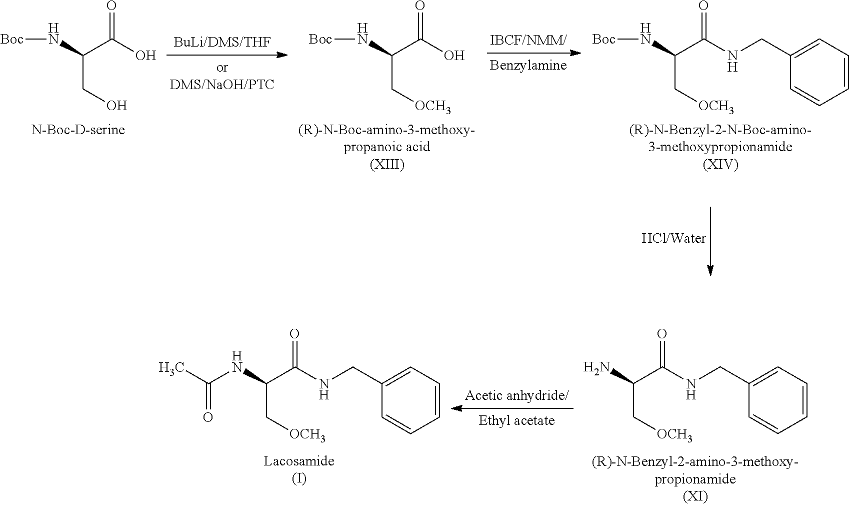 [SMILES: CC(C)(C)OC(=O)N[C@H](CO)C(=O)O ; COC[C@@H](NC(=O)OC(C)(C)C)C(=O)O ; COC[C@@H](NC(=O)OC(C)(C)C)C(=O)NCc1ccccc1 ; COC[C@@H](NC(C)=O)C(=O)NCc1ccccc1 ; COC[C@@H](N)C(=O)NCc1ccccc1]

US 2009/0143472 discloses a process for the preparation of Lacosamide of Formula (I), by reacting N-trityl-D-serine (XV) with methyl iodide in THF to produce O-methyl-N-trityl-D-serine (XVI), which is further reacted with benzylamine in the presence of isobutyl chloroformate (IBCF) and N-methylmorpholine (NMM) to produce N-benzyl-O-methyl-N-trityl-D-serinamide (XVII). Compound (XVII) is converted to N-benzyl-2-amino-3-methoxypropionamide (XI) in the presence of HCl in MDC. Compound (XI) is further reacted with acetic anhydride in dimethylaminopyridine to produce Lacosamide of Formula (I).

The process is as shown in Scheme-VI below:

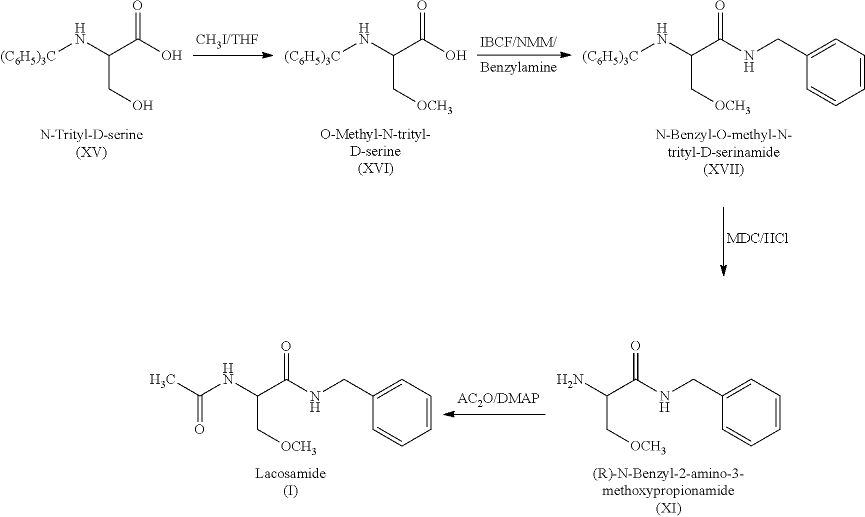

US '472 also discloses a variant process for the preparation of Lacosamide of Formula (I), by reacting N-trityl-D-serine with benzylamine in the presence of isobutyl chloroformate and N-methylmorpholine to produce N-benzyl-N-trityl-D-serinamide (XVIII). Compound (XVIII) is reacted with methyl iodide in THF to produce N-benzyl-O-methyl-N-trityl-D-serinamide (XVII), which is further converted to N-benzyl-2-amino-3-methoxypropionamide (XI) in the presence of HCl in MDC. Compound (XI) is further reacted with acetic anhydride in dimethylamino pyridine to produce Lacosamide of Formula (I).

The process is as shown in Scheme-VII below:

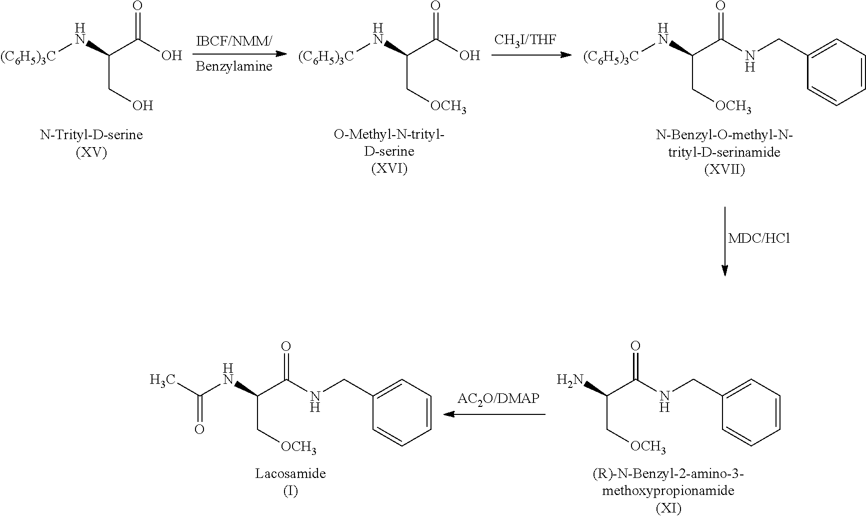

US 2009/0298947 A1 discloses crystalline Form-I, Form-II and amorphous Forms of Lacosamide.

There is a need to develop cost effective and commercially viable process, which provides Lacosamide with high selectivity and without racemisation.

Further, there is a need to develop a purification process, which reduces the unwanted impurities to a pharmaceutically acceptable limit, which inturn provides Lacosamide of high purity with improved yield.

The present invention is specifically directed towards a process, wherein O-methylation of N-benzyl-N-protected-amino-3-hydroxypropionamide of formula (XX), followed by deprotection and N-acetylation to produce Lacosamide of Formula (I). The present invention provides Lacosamide with high purity and yield without racemisation.

The present invention further directed to an improved method for O-methylation of (R)—N-benzyl-2-acetylamino-3-hydroxypropionamide of formula V in a selective manner.

The instant invention also describes a purification process using specific solvents selected from water, isopropyl acetate and there mixtures thereof, which results in pure crystalline Lacosamide Form I.

### SUMMARY OF THE INVENTION

The present invention provides an improved process for the preparation of Lacosamide of Formula I,

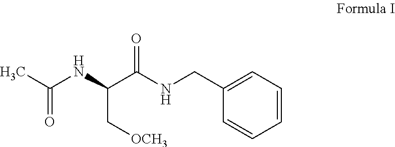 [SMILES: COC[C@@H](NC(C)=O)C(=O)NCc1ccccc1]

comprising O-methylating a compound of Formula (V) in the presence of a methylating agent and a base to produce Lacosamide of Formula (I);

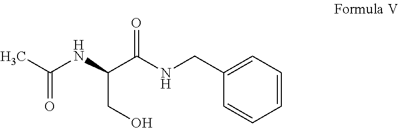 [SMILES: CC(=O)N[C@H](CO)C(=O)NCc1ccccc1]

with proviso that the O-methylation is not carried out in the presence of silver oxide.

Another embodiment of the present invention provides an improved process for the preparation of compound of Formula V, comprising the steps of:
(i) reacting a compound of Formula XIX;

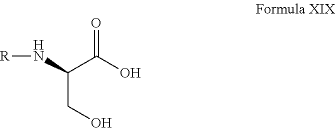

wherein, R represents N-protecting group;
with benzylamine in the presence of a base and an activator of the carboxyl group in a solvent to produce a compound of Formula (XX);

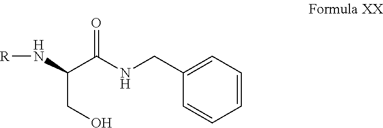

(ii) deprotecting the compound of Formula (XX) in the presence of acid in a solvent to produce a compound of Formula (IV);

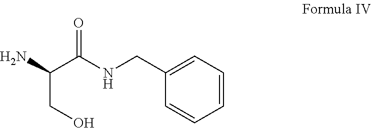 [SMILES: N[C@H](CO)C(=O)NCc1ccccc1]

(iii) acetylating the compound of Formula (IV) in the presence of or absence of a base to produce compound of Formula (V).

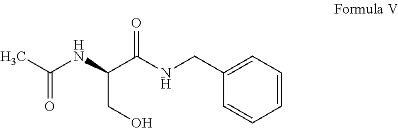 [SMILES: CC(=O)N[C@H](CO)C(=O)NCc1ccccc1]

Another embodiment of the present invention provides an improved process for the preparation of Lacosamide of Formula I,

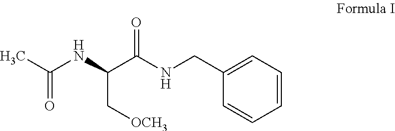 [SMILES: COC[C@@H](NC(C)=O)C(=O)NCc1ccccc1]

which comprises:
   (i) O-methylation of a compound of Formula (XX);

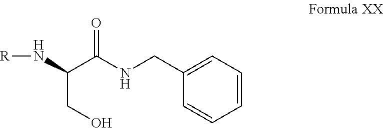

in the presence of a methylating agent and a base to produce Formula (XXI);

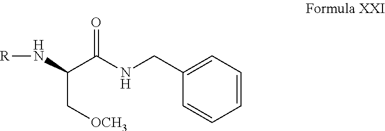

with proviso that the O-methylation is not carried out in the presence of silver oxide.
(ii) deprotecting the compound of Formula (XXI) to produce a compound of Formula (XI);

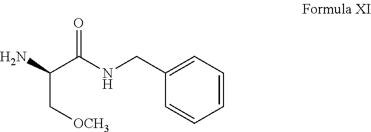 [SMILES: COC[C@@H](N)C(=O)NCc1ccccc1]

(iii) acetylating the compound of Formula (XI) to produce Lacosamide of Formula (I).

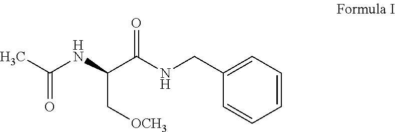 [SMILES: COC[C@@H](NC(C)=O)C(=O)NCc1ccccc1]

Another embodiment of the present invention provides a process for the preparation of Lacosamide of Formula (I),
which comprises:
   (i) reacting a compound of Formula (XXII),

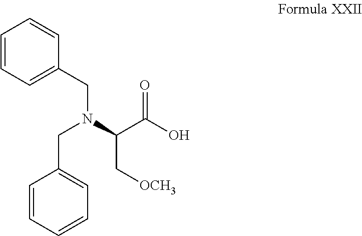 [SMILES: COC[C@H](C(=O)O)N(Cc1ccccc1)Cc1ccccc1]

with benzylamine in presence of a base and an activator of the carboxyl group to produce a compound of Formula (XXIII),

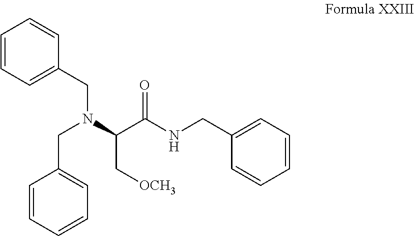 [SMILES: COC[C@H](C(=O)NCc1ccccc1)N(Cc1ccccc1)Cc1ccccc1]

(ii) deprotecting the compound of Formula (XXIII) to produce a compound of Formula (XI),

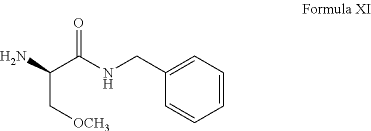 [SMILES: COC[C@@H](N)C(=O)NCc1ccccc1]

(iii) acetylating the compound of Formula (XI) to produce Lacosamide of Formula (I).

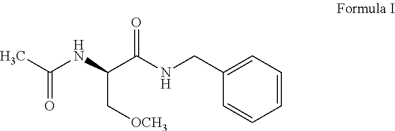 [SMILES: COC[C@@H](NC(C)=O)C(=O)NCc1ccccc1]

According to another embodiment, the present invention also provides a process for the purification of Lacosamide of Formula I, comprises:
(i) preparing a solution of crude Lacosamide in a solvent selected from water;
(ii) optionally, filtering the solution of step (i);
(iii) precipitating Lacosamide Form I by cooling the solution;
(iv) isolating pure Lacosamide in crystalline Form I.

According to an embodiment, the present invention also provides a process for the purification of Lacosamide of Formula I, comprises:
(i) suspending crude Lacosamide in a solvent selected from isopropyl acetate, di-n-butyl ether and mixtures thereof;
(ii) isolating pure Lacosamide in crystalline Form I.

### DETAILED DESCRIPTION OF THE INVENTION

In one embodiment of the invention, the present invention further provides an improved process for the preparation of (R)-2-acetamido-N-benzyl-3-methoxypropionamide (Lacosamide) of Formula I.

The process comprises, reacting N-protected D-serine (XIX) with benzylamine in the presence of a suitable base and an activator of carboxyl group in a suitable solvent to produce a compound of formula (XX.) N-protecting group of the compound of the formula (XIX) is selected from benzyl, allyl, phenacyl, acetoxypropyl, methoxymethyl, benzyloxymethyl, pivaloyloxymethyl, tertrahydropyranyl, 2,4-dinitrophenyl, o-nitrobenzyl, di(p-methoxyphenyl)methyl, triphenylmethyl (trityl), (p-methoxyphenyl)diphenylmethyl, diphenyl-4-pyridylmethyl, 2-picolyl N-oxide, N′-isopropylidene, benzylidene, p-nitrobenzylidene, salicylidene, (5,5-dimethyl-3-oxo-1-cyclohexenyl), diphenylphosphinyl, dimethyl-thiophosphinyl, benzensulfenyl, o-nitrobenzenesulfenyl, 2,4,6-trimethylbenzenesulfonyl, toluenesulfonyl, benzylsulfonyl, trifluoromethylsulfonyl, phenacylsulfonyl; carbamates such as methylcarbamate, 1,1-dimethylpropynyl carbamate, 1-methyl-1-phenylethylcarbamate, 1-methyl-1-(4-biphenylyl)ethyl carbamate, 1,1-dimethyl-2-haloethylcarbamate,1,1-dimethyl-2-cyanoethyl carbamate, t-butoxycarbonyl (Boc), trichloro-t-butoxycarbonyl (TCBoc), cyclobutylcarbamate, 1-methylcyclobutylcarbamate, vinyl carbamate, 8-quinolyl carbamate, N-hydroxypiperidinyl carbamate, 4,5-diphenyl-3-oxolin-2-one, carbobenzoxy (Cbz), 9-fluorenyl methyloxycarbonyl (9-Fmoc), 3,4-dimethoxy-6-nitrobenzyl carbomate, 2,4-dichlorobenzyl carbomate, 5-benzisoxazolylmethyl carbomate, 9-anthrylmethyl carbamate, isonicotinyl carbamate, S-benzylcarbamate, N—(N′-phenylaminothiocarbonyl)derivative, p-nitrobenzyloxycarbonyl, p-methoxybenzyloxycarbonyl, trityloxycarbonyl(triphenylmethyloxycarbonyl), diphenylmethyloxycarbonyl, 1-adamantyloxycarbonyl, cinnamyloxycarbonyl, N-hydroxy piperidinyloxycarbonyl, 2-trimethylsilylethyloxycarbonyl, 2,2,2-trichloroethoxycarbonyl (Troc), allyloxycarbonyl (Alloc); amides such as formyl, acetyl, chloroacetyl, trichloroacetyl, trifluoroacetyl, o-nitrophenylacetyl, o-nitrophenoxyacetyl, acetoacetyl, 3-phenylpropionyl, 3-(p-hydroxyphenyl)propionyl, 2-methyl-2-(o-nitrophenoxy)propionyl, 2-methyl-2-(o-phenylazophenoxy)propionyl, 4-chlorobutyryl, o-nitrocinnamoyl, picolinoyl, (N-acetylmethionyl), benzoyl, phthaloyl, dithiasuccinoyl.

The suitable base used in the above reaction is selected from triethylamine, diisopropylethylamine, 1,8-diazabicyclo[5.4.0]undec-7-ene, 1,4-diazabicyclo-[2.2.2]octane, potassium bicarbonate, potassium carbonate, sodium carbonate, sodium bicarbonate, morpholine derivative, preferably 4-methylmorpholine and an activator of the carboxyl group is selected from carbodiimide, N,N-carbonyldiimidazole N,N-dimethylaminopyridine(DMAP) or an alkyl chloroformate, preferably isobutyl chloroformate.

The suitable organic solvents used in the above reaction are selected from halogenated solvents, such as dichloromethane, ethylene dichloride, and chloroform; ether, toluene, ethyl acetate or mixture thereof. The reaction is performed at a temperature ranging from −15° C. to about 35° C. based on the solvent or mixture of solvents used for the reaction. The activator of the carboxyl group and base are added to the solution of compound of Formula (XIX) in the organic solvent. More preferably, the reagent is added slowly in a drop-wise manner. Most preferably, this addition is carried out while maintaining the reaction mixture at a temperature of about −15° C. to about 5° C. The sufficient period of time necessary for obtaining compound (XX) will depend on the parameters of the reaction. Preferably, maintaining the reaction mixture for about 1 to about 6 hours. More preferably, the reaction mixture is maintained for 1 hour to about 2 hours.

The compound (XX) obtained by the above process is isolated by precipitation of compound from the reaction mixture or by removing the solvent from the reaction mixture.

Compound of Formula (XX) is then subjected to de-protection to produce a compound of Formula (XI). The de-protection of N-protecting group is performed using the conditions suitable for the protecting agents. When the N-protecting group is benzyl, the de-protection reaction is carried out using hydrogenation catalyst selected from Pd/C, Pd(OH)₂/C in a suitable solvent. The suitable solvent is selected from alcohol such as methanol, ethanol, isopropanol, etc.

When the de-protecting group is trityl, the de-protection is carried out using acid selected from hydrochloric acid, hydrobromic acid, acetic acid etc; base selected from alkalimetal hydroxides such as sodium hydroxide, potassium hydroxide and cesium hydroxide, etc.

When the protecting group is carbamate, the de-protection reaction is carried out using mild acidic conditions in a suitable solvent. Mild acids such as acetic acid, oxalic acid, tartaric acid, phosphoric acid (H₃PO₄), sodium hydrogen phosphate (Na₂HPO₄), etc. or strong acid e.g., hydrochloric acid, sulphuric acid, trifluoroacetic acid, etc. are used. The suitable solvent is selected from aromatic solvents selected from toluene, xylene, etc., and aliphatic solvents selected from chlorinated solvents dichloromethane, chloroform; alcohols such as methanol, ethanol, isopropanol; ethyl acetate, cyclopentyl methyl ether, etc. are used for the de-protection step. Basic compounds are used to neutralize the reaction medium. A solution of strong or mild basic compounds is suitable for neutralization. Some examples of these basic compounds are ammonia, ammonium hydroxide, ammonium carbonate, ammonium bicarbonate sodium bicarbonate, sodium carbonate, sodium hydroxide, potassium bicarbonate, potassium carbonate, potassium hydroxide, calcium bicarbonate, calcium hydroxide, calcium carbonate, magnesium hydroxide, magnesium carbonate, magnesium bicarbonate, etc.

The acid is added to the solution of compound of Formula (XX) in organic solvent and the de-protection reaction is allowed to proceed for about 1 hour at 15 to 40° C., preferably for 20-50 minutes at 20-35° C., most preferably for 30-40 minutes at 25-30° C. Also, the reaction is performed at higher or lower temperatures such as any temperature between 15 and 40° C. if the reaction time is adapted accordingly. After completion of the reaction, a solution of the base compound is added to the reaction mixture.

When the de-protecting group is t-butyloxycarbonyl (Boc), hydrochloric acid used as de-protecting agent, ethyl acetate, dichloromethane or ethanol as organic solvent and aqueous sodium hydroxide or potassium hydroxide as base is used for neutralization. The de-protected compound of (R)—N-benzyl-2-amino-3-hydroxypropionamide of Formula (IV) is then isolated from the reaction mixture and optionally purified.

Compound of Formula (IV) is acetylated to produce the compound of Formula V. Acetic anhydride, acetyl chloride, acetic acid or the like and derivatives thereof is used as an acetylating agent. The acetylation is performed in the presence or absence of a base. The base is selected from triethylamine, pyridine, dimethylaminopyridine, N-Methylmorpholine. The acetylation reaction is performed in presence of a solvent selected from dichloromethane, toluene, ethyl acetate, water.

Base is added to the solution of compound of Formula (IV) in an organic solvent and acetylating agent is then slowly added to the mixture. The reaction is allowed to proceed for up to 2 hours at temperature ranging from 5 to 40° C. Compound of Formula (V) is then isolated from the reaction mixture and purified.

Acetic anhydride as acetylating agent, dichloromethane, chloroform, ethyl, acetate, isopropyl acetate or water as solvent and pyridine as base is used for the acetylation.

O-methylation of a compound (V) is performed in the presence of methylating agent to produce a Lacosamide of Formula I. Methylating agents selected from methyl halide such as methyl iodide, methyl chloride, methyl bromide, methyl fluoride; dimethyl sulfate, trimethyl silyldiazomethane, dimethyl sulfoxide (DMSO) is used for this reaction. The most preferred methylating agent is dimethyl sulfate. The reaction is performed in the presence of a base, which is selected from hydride, hydroxide and/or oxides of metals such as hydride, carbonates, hydroxide and/or oxides of sodium, potassium and calcium. The most preferred base is sodium or potassium hydroxide.

The O-methylation is optionally be performed in the presence of a phase transfer catalyst (PTC), selected from tetraethylammonium-p-toluenesulfonate, tetrapropylammonium trifluoromethane sulfonate, tetraphenylphosphonium hexafluoroantimonate, acetylpyridinium bromide, triphenylmethyl triphenylphosponium chloride, benzyltriethylammonium chloride, benzyltrimethylammonium chloride, benzyltriphenylphosphonium chloride, benzytributylammonium chloride, butyltriethylammonium bromide, butyltriphenylphosphonium bromide, cetyltrimethyl ammonium bromide, cetyltrimethyl ammonium chloride, ethyltriphenylphosphonium bromide, ethyltriphenylphosphonium iodide, methyltrioctylammonium bromide, methyltriphenylphosphonium bromide, methyltriphenylphosphonium iodide, phenyltrimethylammonium chloride, tetrabutylammonium hydroxide, tetrabutylammonium perchlorate, tetrabutylammonium bromide, tetrabutylammonium hydrogensulphate, tetrabutylammonium iodide, tetrabutylammonium tetrafluoroborate, tetrabutylammonium thiocyanate, tetraethylammonium hydroxide, tetraethylammonium iodide, tetraethylammonium bromide, tetramethylammonium chloride, tetramethyl-ammonium iodide, tetramethylammonium chloride, tetraoctylammonium bromide, tetraphenylphosphonium bromide, tetrapropylammonium hydroxide, tetrapropylammonium bromide and tributylmethylammonium chloride, wherein tetrabutylammonium salts and particularly tetrabutylammonium halides, e.g. the bromide are especially preferred.

The solvents used for the O-methylation reaction are selected from tetrahydrofuran (THF), dichloromethane (MDC), dimethyl sulfoxide (DMSO), acetonitrile (MeCN), ethyl acetate, monoglyme and diglyme or mixture thereof.

The methylating agent is added to the mixture containing compound of formula (V), base and an organic solvent. The reaction is usually allowed to proceed for at least 2 hours at −10 to 20° C., and preferably for 2.5-5 hours at 0 to 10° C. Also, the reaction is performed at higher or lower temperatures such as any temperature between −20 and 20° C. if the reaction time is adapted accordingly. Lacosamide of Formula (I) is then isolated from the mixture and optionally purified.

Compound of Formula XIX used in the present invention is prepared by the known methods in the art by reacting D-serine with a protecting reagent under suitable reaction conditions.

In another embodiment, the present invention further provides an alternative process for the preparation of Lacosamide of Formula (I).

The process comprises, N-protected D-serine (XIX) with benzylamine in the presence of a suitable base and an activator of carboxyl group in a suitable solvent to produce compound of formula (XX). N-protecting group, of the compound of formula (XIX) is selected from t-butoxycarbonyl (Boc), carbobenzoxy (Cbz), 9-fluorenyl methyloxycarbonyl (9-FMOC).

The suitable base used in the above reaction is selected from triethylamine, diisopropylethylamine, 1,8-diazabicyclo[5.4.0]undec-7-ene, 1,4-diazabicyclo-[2.2.2]octane, potassium bicarbonate, potassium carbonate, sodium carbonate, sodium bicarbonate, morpholine derivative, preferably 4-methylmorpholine and an activator of the carboxyl group is selected from carbodiimide, N,N-carbonyldiimidazole N,N-dimethylaminopyridine(DMAP) or an alkyl chloroformate, preferably isobutyl chloroformate.

The suitable organic solvents for the above reaction are selected from halogenated solvents, such as dichloromethane, ethylene dichloride, and chloroform; ether, toluene, ethyl acetate. The reaction is performed at a temperature ranging from −10° C. to about 35° C. based on the solvent or mixture of solvents used for the reaction. The activator of the carboxyl group and base are added to the solution of compound of Formula (XIX) in the organic solvent. More preferably, the reagent is added slowly in a drop-wise manner. Most preferably, this addition is while maintaining the reaction mixture at a temperature of about −10° C. to about 5° C. The sufficient period of time necessary for obtaining compound (XX) will depend on the parameters of the reaction. Preferably, maintaining the reaction mixture for' about 1 to about 6 hours. More preferably, the reaction mixture is maintained for 1 hour to about 2 hours.

The compound (XX) obtained by the above process is used as such in the next step or isolated by precipitation of compound from the reaction mixture or by removing the solvent from the reaction mixture.

O-methylation of a compound (XX) is performed in the presence of methylating agent to produce a compound of Formula (XXI). Methylating agent is selected from methyl halide such as methyl iodide, methyl chloride, methyl bromide, methyl fluoride; dimethyl sulfate, trimethyl silyldiazomethane, dimethyl sulfoxide (DMSO). The most preferred methylating agent is dimethyl sulfate. The reaction is performed in the presence of a base, which is selected from hydride, hydroxide and/or oxides of metals such as hydride, carbonates, hydroxide and/or oxides of sodium, potassium and calcium. The most preferred base is sodium or potassium hydroxide.

The O-methylation can optionally be performed in the presence of a phase transfer catalyst (PTC), selected from tetraethylammonium-p-toluenesulfonate, tetrapropylammonium trifluoromethane sulfonate, tetraphenylphosphonium hexafluoroantimonate, acetylpyridinium bromide, triphenylmethyl triphenylphosponium chloride, benzyltriethylammonium chloride, benzyltrimethylammonium chloride, benzyltriphenylphosphonium chloride, benzytributylammonium chloride, butyltriethylammonium bromide, butyltriphenylphosphonium bromide, cetyltrimethyl ammonium bromide, cetyltrimethyl ammonium chloride, ethyltriphenylphosphonium bromide, ethyltriphenylphosphonium iodide, methyltrioctylammonium bromide, methyltriphenylphosphonium bromide, methyltriphenylphosphonium iodide, phenyltrimethyl ammonium chloride, tetrabutylammonium hydroxide, tetrabutylammonium perchlorate, tetrabutylammonium bromide, tetrabutylammonium hydrogensulphate, tetrabutylammonium iodide, tetrabutylammonium tetrafluoroborate, tetrabutylammonium thiocyanate, tetraethylammonium hydroxide, tetraethylammonium iodide, tetraethylammonium bromide, tetramethylammonium chloride, tetramethylammonium iodide, tetramethylammonium chloride, tetraoctylammonium bromide, tetraphenylphosphonium bromide, tetrapropylammonium hydroxide, tetrapropylammonium bromide and tributylmethylammonium chloride, wherein tetrabutylammonium salts and particularly tetrabutylammonium halides, e.g. the bromide are especially preferred.

The solvents that are used for the O-methylation reaction are generally organic solvents selected from tetrahydrofuran (THF), dichloromethane (MDC), dimethyl sulfoxide (DMSO), acetonitrile (MeCN), ethyl acetate, acetone, 1,2-dimethoxy ethane, monoglyme, diglyme or mixture thereof.

The methylating agent is added to the mixture containing compound of formula (XX), base and an organic solvent. The reaction is usually allowed to proceed for at least 2 hours at −10 to 20° C., and preferably for 2.5-5 hours at 0 to 10° C. Also, the reaction is performed at higher or lower temperatures such as any temperature between −20 and 20° C. if the reaction time is adapted accordingly. The O-methylated compound of Formula (XXI) is then isolated from the mixture and optionally purified.

Compound of Formula (XXI) is then subjected to de-protection to produce a compound of Formula (XI). The de-protection reaction is performed using de-protecting agents selected from acids. Strong as well as mild acidic conditions are suitable for the de-protection reaction. Mild acids such as acetic acid, oxalic acid, tartaric acid, phosphoric acid (H₃PO₄), sodium hydrogen phosphate (Na₂HPO₄), etc. or strong acid e.g., hydrochloric acid, sulphuric acid, trifluoroacetic acid, etc. are used. Organic solvent is selected from aromatic or aliphatic solvent. Aromatic solvents such as toluene, xylene, etc., and aliphatic solvents such as chlorinated solvents dichloromethane, chloroform; alcohols such as methanol, ethanol, isopropanol; ethyl acetate, cyclopentyl methyl ether are used in de-protection step. Basic compounds are used to neutralize the reaction medium. A solution of strong or mild basic compounds are suitable for neutralization. Some examples of these basic compounds are ammonia, ammonium hydroxide, ammonium carbonate, ammonium bicarbonate sodium bicarbonate, sodium carbonate, sodium hydroxide, potassium bicarbonate, potassium carbonate, potassium hydroxide, calcium bicarbonate, calcium hydroxide, calcium carbonate, magnesium hydroxide, magnesium carbonate, magnesium bicarbonate, etc.

Accordingly, the acid is added to the solution of compound of Formula (XXI) in organic solvent and the de-protection reaction is allowed to proceed for about 1 hour at 15 to 40° C., preferably for 20-50 minutes at 20-35° C., most preferably for 30-40 minutes at 25-30° C. Also, the reaction is performed at higher or lower temperatures such as any temperature between 15 and 40° C. if the reaction time is adapted accordingly. After completion of the reaction, a solution of the base compound is added to the reaction mixture.

Hydrochloric acid as de-protecting agent, dichloromethane or ethanol as organic solvent and aqueous sodium hydroxide or potassium hydroxide as base are used for neutralization. The de-protected compound of (R)—N-benzyl-2-amino-3-methoxypropionamide of Formula (XI) is then isolated from the reaction mixture and optionally purified.

Compound of Formula (XI) is acetylated to produce the Lacosamide of Formula I. Acetic anhydride, acetyl chloride, acetic acid or the like and derivatives thereof are used as an acetylating agent. The acetylation is performed in the presence or absence of a base. The base used is nitrogen-containing base selected from pyridine, dimethylaminopyridine, sodium acetate. The acetylation reaction is performed in presence of solvent selected from dichloromethane, toluene, ethyl acetate, water or mixture thereof.

Base is added to the solution of compound of Formula (XI) in an organic solvent and acetylating agent is then slowly added to the mixture. The reaction is allowed to proceed for up to 2 hours at temperature ranging from 5 to 40° C. Lacosamide produced is then isolated from the reaction mixture and purified.

Acetic anhydride as acetylating agent, dichloromethane, chloroform, ethyl acetate, isopropyl acetate or water as solvent and pyridine as base is used for the acetylation.

In another embodiment, the present invention further provides an alternative process for preparation of Lacosamide of Formula (I).

The process comprises, reacting a compound of formula (XXII) with benzylamine in the presence of suitable base and an activator of carboxyl group in a suitable solvent to produce a compound of formula (XXIII).

The suitable base used in the above reaction is selected from triethylamine, diisopropylethylamine, 1,8-diazabicyclo[5.4.0]undec-7-ene, 1,4-diazabicyclo[2.2.2]octane, potassium bicarbonate, potassium carbonate, sodium carbonate, sodium bicarbonate, morpholine derivative, preferably 4-methylmorpholine and an activator of the carboxyl group is selected from carbodiimide, N,N-carbonyldiimidazole, N,N-dimethylaminopyridine(DMAP) or an alkyl chloroformate, preferably isobutyl chloroformate.

The suitable organic solvents for the above reaction is selected from halogenated solvents, such as dichloromethane, ethylene dichloride, and chloroform; ether, toluene, ethyl acetate. The reaction is performed at a temperature ranging from −10° C. to about 35° C. based on the solvent or mixture of solvents used for the reaction. The activator of the carboxyl group and base are added to the solution of compound of Formula (XXI) in the organic solvent. More preferably, the reagent is added slowly in a drop-wise manner. Most preferably, this addition is carried out while maintaining the reaction mixture at a temperature of about −10° C. to about 5° C. The sufficient period of time necessary for obtaining compound (XXII) will depend on the parameters of the reaction. Preferably; maintaining the reaction mixture for about 1 to about 6 hours. More preferably, the reaction mixture is maintained for 1 hour to about 2 hours.

The compound (XXIII) obtained by the above process is isolated by precipitation of compound from the reaction mixture or by removing the solvent from the reaction mixture.

Compound of Formula (XXIII) is reduced in the presence of hydrogenation catalyst selected from Pd/C, Pd(OH)₂/C in a suitable solvent to produce a compound of Formula (XI). The suitable solvent is selected from alcohol such, as methanol, ethanol and isopropanol.

Compound of Formula (XI) is acetylated to produce the Lacosamide of Formula I. Acetic anhydride, acetyl chloride, acetic acid or the like and derivatives thereof are used as an acetylating agents. The acetylation is performed in the presence or absence of a base. The base is selected from triethylamine, pyridine, dimethylaminopyridine, N-Methylmorpholine. The acetylation reaction is performed in presence of solvent selected from dichloromethane, toluene, ethyl acetate, water.

Base is added to the solution of compound of Formula (XI) in an organic solvent and acetylating agent is then slowly added to the mixture. The reaction is allowed to proceed for up to 2 hours at temperature ranging from 5 to 40° C. Lacosamide of Formula (I) is then isolated from the reaction mixture and purified.

Acetic anhydride as acetylating agent, dichloromethane or water as solvent and pyridine as base is used for the acetylation.

The compound of formula XXII is prepared according to the process disclosed in JP 2010/37206 A1.

In another embodiment, the present invention further provides a process for the purification of Lacosamide of Formula (I).

The process comprises dissolving crude Lacosamide in a solvent selected from water, precipitating Lacosamide Form I by cooling the solution to about 0-5° C., and isolating crystalline Lacosamide Form I.

Optionally, the crude Lacosamide is dissolved in a solution at a temperature from about 65 to 70° C. to about the boiling temperature of the solvent. Optionally, the Lacosamide Form I is isolated by filtration. Optionally, the solution is treated with carbon, followed by filtration to remove insoluble material. The step of cooling the reaction is performed by cooling the solution to a temperature from about −10° C. to 25° C. temperature. The solvent is selected from water, isopropyl acetate and mixtures thereof.

In another embodiment, the present invention further provides an alternative process for the purification of Lacosamide of Formula (I).

The process comprises suspending crude Lacosamide in a solvent selected from dichloromethane, isopropyl acetate, di-n-butyl ether and mixtures thereof, stirring the resulting suspension at about 40-50° C., and isolating crystalline Lacosamide Form I. Optionally, the Lacosamide Form I is isolated by filtration.

It has been observed that purification of crude Lacosamide using above solvents results in pure crystalline Lacosamide Form I, having (S)-enantiomer less than 0.1% by HPLC analysis.

The following examples are provided to illustrate the invention and are merely for illustrative purpose only and should not be construed to limit the scope of the invention.

### EXAMPLE-1

### Step 1

### Preparation of tert-Butyl-N-[(1R)-2-(Benzylamino)-1-(hydroxymethyl)-2-oxo-ethyl]carbamate (N-Boc-D-serinamide)

N-Boc-D-Serine (32.8 g, 0.1599 m) was suspended in methylene chloride (160 ml) was cooled to ←5° C. Isobutyl chloroformate (22.3 g, 0.1632 m) was added to the above suspension at a temperature ←5° C. and the resultant mixture was aged for 5-10 min at ←5° C. N-Methyl morpholine (16.5 g, 0.1631 m) was added in 10-15 min at ←5° C. The resultant solution was aged for 30-40 min at ←5° C. Benzylamine (17.7 g, 0.1652 m) was added at ←5° C. in 10-15 min. The mixture was aged for 70-80 min at <0° C., followed by successively washed with water (70 ml), 1N HCl (70 ml), 8% sodium bicarbonate (70 ml) and DM water (70 ml) produced crude (R)—N-benzyl-2-N-Boc-amino-3-hydroxypropionamide. It is purified from n-hexane.

HPLC purity: ˜99%, Chiral Purity 99%. Yield: 34 g.

### Step 2

### Preparation of (2R)-2-Amino-N-benzyl-3-hydroxy-propanamide (D-Serinamide hydrochloride)

Boc-D-serinamide (40 g, 0.1360 m) was suspended in ethyl acetate (120 ml) at 25-30° C. Ethyl acetate hydrochloride (18%, 75 g) was added at 25-30° C. and stirred the reaction mass at 25-30° C., slowly compound crystallized out. The suspension was stirred for 60-70 min at 25-30° C. The compound was filtered and washed with ethyl acetate (2×30 ml). The compound was dried at 40-50° C. under reduced pressure to produce (2R)-2-amino-N-benzyl-3-hydroxy-propanamide. Purity: 30.5 g.

### Step 3

### Preparation of (2R)-2-acetamido-N-benzyl-3-hydroxypropanamide (Acetyl-D-serin-amide)

D-serinamide hydrochloride (20 g, 0.08676 m) was suspended in methylene chloride (130 ml) at 25-30° C. Triethylamine (8.9 g, 0.08795 m) was added and stirred the mass for 40-50 min at 25-30° C. Acetic anhydride (8.9 g, 0.08668 m) was added at 25-30° C. The mass was stirred for 2 h at 25-30° C. and then compound slowly crystallized but. Further, the suspension was stirred for another 2-3 h at 25-30° C. The compound was filtered and washed with methylene chloride (2×10 ml). The compound was dried, at 40-50° C. under reduced pressure till to produce (2R)-2-acetamido-N-benzyl-3-hydroxypropanamide.

Yield: 12.0 g, Chromatographic purity: ˜99%.

### Step 4

### Preparation of (R)—N-Benzyl-2-acetamido-3-methoxypropionamide (Lacosamide)

Acetyl D-serinamide (10 g, 0.04233) was suspended in methylene chloride (100 ml) at 25-30° C. Potassium hydroxide (3.56, 0.06344 m) was added and the reaction mass was stirred for 20-30 min at 25-30° C. Dimethyl sulfate (6.4 g, 0.05074 m) was added at 25-30° C. in 10-15 min and the mass was stirred for 2-3 h at 25-30° C. Then the reaction mass was poured into citric acid solution at 10-15° C. The organic layer was separated and washed with DM water (30 ml). The organic layer was separated and methylene chloride was distilled off at 35-40° C. under reduced pressure to get solid compound (Lacosamide crude). The crude compound was suspended in isopropyl acetate (63 ml) at 25-30° C. The suspension was stirred for 60-70 min at 25-30° C. The compound was filtered and washed with isopropyl acetate (2×10 ml). The compound was dried at 40-50° C. under reduced pressure to Lacosamide.

Yield: 5.6 g, Chiral purity: 99.95%.

### EXAMPLE-2

### Step 1

### Preparation of (R)—N-benzyl-2-N-Boc-amino-3-hydroxypropionamide

### Method a

N-Boc-D-Serine (32.8 g, 0.1599 mol) was suspended in methylene chloride (160 ml) and cooled to ←5° C. Isobutyl chloroformate (22.3 g, 0.1632 mol) was added to the above suspension at a temperature ←5° C. and the resultant mixture was aged for 5-10 min at ←5° C. N-Methyl morpholine (16.5 g, 0.1631 mol) was added in 10-15 min at ←5° C. The resultant solution was aged for 30-40 min at ←5° C. Benzyl amine (17.7 g, 0.1652 mol) was added at ←5° C. in 10-15 min. The mixture was aged for 70-80 min at <0° C., followed by successively washed with water (70 ml), 1N HCl (70 ml), 8% sodium bicarbonate (70 ml) and DM water (70 ml) to produce (R)—N-benzyl-2-N-Boc-amino-3-hydroxypropionamide. HPLC purity: ˜87%, Chiral Purity 99.2%.

### Method b

N-Boc-D-Serine (40 g, 0.1949 mol) was suspended in methylene chloride (125 ml) and cooled to <5° C. N-methylmorpholine (20.5 g, 0.2006 mol) was added in 5-10 min at <5° C. and the resulting mixture was aged for 10-15 min to get clear solution. The solution was added to a solution of isobutyl chloroformate (27.4 g, 0.2006 mol) in methylene chloride (125 ml) at ←10° C. in 60-70 min. The resulting solution was aged for 10-15 min at ←8° C. and benzyl amine (21.3 g, 0.1987 m) was added at −5° C. to 0° C. in 20-30 min and aged for 60-70 min at ←5° C. to 0° C., followed by successively washed with water (80 ml), 1N HCl (80 ml), 8% sodium bicarbonate (80 ml) and DM water (80 ml) to produce (R)—N-Benzyl-2-N-Boc-amino-3-hydroxy-propionamide in methylene chloride. HPLC purity: 93.31%, Chiral Purity 99.5%.

### Step 2

### Production of (R)—N-benzyl-2-N-Boc-amino-3-methoxypropionamide

(R)—N-Benzyl-2-N-Boc amino-3-hydroxy propionamide solution prepared as per the method b was cooled to <5° C. and potassium hydroxide (17.6 g, 0.3136 mol) was added at <5° C. The resulting suspension was aged for 5-10 min at <5° C. and dimethyl sulfate (29.6 g, 0.2346 mol) was added at <5° C. in 10-15 min. The resulting mixture was aged for 3-5 h at <5° C. Water (80 ml) was added to the suspension and separated the phases. The organic layer was washed with a solution of citric acid (20.0 g) in DM water (80 ml) to produce (R)—N-benzyl-2-N-Boc-amino-3-methoxypropionamide solution in methylene chloride with HPLC purity (90%), chiral purity (98%). The purity of the crude compound is optionally improved by, crystallization from a mixture of hexane and ethyl acetate.

### Step 3

### Production of (R)-2-amino-N-benzyl-3-methoxypropionamide

(R)—N-Benzyl-2-N-Boc-amino-3-methoxypropionamide solution in methylene chloride was concentrated till the solution volume was half of the original volume at 35-40° C. under reduced pressure. Hydrochloric acid (36%, 55 ml, 0.6137 mol) was added at 25-30° C. and the resulting mixture was aged for 60-90 min at 25-30° C. Water (60 ml) was added and the phases were separated. The aqueous phase was washed with methylene chloride (50 ml). The aqueous layer was basified to pH 10-11 with 30% sodium hydroxide at 25-30° C. and saturated with sodium chloride (˜20.0 g). The aqueous layer was extracted with methylene chloride (2×100 ml) and the combined organic layer was dried over anhydrous sodium sulfate. Methylene chloride was distilled off at 35-40° C. under diminished pressure to get oily mass. Yield: 40.0 g, HPLC purity: ˜94%, Chiral purity: >98%.

### Step 4

### Preparation of (R)—N-Benzyl-2-acetamido-3-methoxypropionamide (Lacosamide)

(R)—N-Benzyl-2-amino-3-methoxypropionamide (35 g, 0.1681 mol) was dissolved in DM water (400 ml) at ambient temperature. The solution was cooled to 5-10° C., followed by pyridine (1.76 g, 0.0222 mol) and acetic anhydride (21.5 g, 0.2106 mol) were added at 5-10° C. in 15-20 min. The solution was stirred for 30-40 min at 5-10° C. and raised to room temperature (25-30° C.) in over 30 min. The solution was further stirred for 30-40 min at room temperature (25-30° C.). The compound was extracted with methylene chloride (2×200 ml). The combined organic layer was washed with hydrochloric acid (1N HCl, 70 ml), followed by aqueous sodium bicarbonate solution (8% w/v, 70 ml) and DM water (70 ml) and distilled off MDC under vacuum at 35-40° C. to get solid product. The solid was suspended in isopropyl acetate (200 ml) and stirred the suspension for 60-70 min at room temperature (25-30° C.). Filtered the product and washed with isopropyl acetate. Yield: 30 g, Chiral purity: 99.5%.

### EXAMPLE-3

### Step 1

### Preparation of (R)-methyl-2-(dibenzylamino)-3-hydroxypropanoate (N,N-Dibenzyl-D-Serine methyl ester)

D-Serine methyl ester hydrochloride (50 g, 0.3215 mol) was dissolved in acetonitrile (500 ml) at 25-30° C. and cooled to 10-20° C. Potassium carbonate (200 g, 1.9469 mol) and benzylbromide (110 g, 0.6430 mol) were added at 10-20° C. The temperature of the reaction mass was raised to 25-30° C. and the reaction mass was stirred for 8 h at 25-30° C. Potassium carbonate and wash with acetonitrile (20 ml) were filtered off from the reaction mass and the filtrate was concentrated at 40-50° C.: under reduced pressure to get a crude compound. The crude compound was dissolved in ethyl acetate (250 ml) and triethyl amine (40 ml) was added. The mass was stirred for 60-70 min at 25-30° C. and the resulting reaction mass was filtered and the mother liquors were washed successively with DM water (100 ml) and aqueous sodium chloride solution (30% w/v, 100 ml). The organic layer was separated and dried over by sodium sulfate. Sodium sulfate was filtered off and washed with ethyl acetate (30 ml). The filtrate mother liquors were concentrated at 40-50° C. under reduced pressure to get oily mass. Yield: 38 g (0.76 w/w based on input).

### Step-2

### Preparation of (R)-methyl-2-(dibenzylamino)-3-methoxypropanoate (N,N-dibenzyl-O-methyl D-serine methyl ester)

N,N-dibenzyl D-serine methyl ester (74 g, 0.2471 m) was dissolved in N,N-dimethylformamide (250 ml) at 25-30° C. The solution was cooled to −20° to −15° C. under nitrogen. Sodium hydride (60%, 16.7 g, 0.4946 mol) was added at −20° to −15° C. in 60-70 min under nitrogen. The reaction mass was stirred at −20° to −15° C. over a period of 20-30 min. A solution of methyl iodide (70.2 g, 0.4948 m) in N,N-dimethylformamide (50 ml) was added at −20° to −15° C. in 30-40 min. The reaction mass was stirred at −20° to −15° C. for 2 h and the reaction mass was poured into DM water (500 ml, 0-5° C.). The compound was extracted with ethyl acetate (2×250 ml) and the combined organic layer was washed with water (200 ml), sodium metabisulfite (10% w/v, 2×100 ml) and brine (100 ml, 30% w/v). The organic layer was separated and dried over by sodium sulfate for 10-15 min at 25-30° C. Sodium sulfate was filtered off and washed with ethyl acetate (20 ml). The filtrate mother liquors were distilled at 40-50° C. under reduced pressure to get an yellow oily mass. Yield: 55 g (0.74 w/w, based on input).

### Step 3

### Preparation of (R)-methyl-2-(dibenzylamino)-3-methoxypropanoic acid (N,N-dibenzyl O-methyl D-serine)

N,N-dibenzyl O-methyl D-serine methyl ester (50 g, 0.1597 mol) was dissolved in methanol (225 ml) at 25-30° C. A solution of lithium hydroxide (7.65 g, 0.191 mol) in DM water (25 ml) was added to the above solution at 25-30° C. The reaction mixture was stirred at 25-30° C. for 15 h. The reaction mass was concentrated under reduced pressure at 40-45° C. and DM water (50 ml) was added and the pH of the mass was adjusted to 3.0±0.1 with aqueous hydrochloric acid (5N). The compound was extracted with ethyl acetate (2×250 ml) at 25-30° C. The combined organic layer was dried over by sodium sulfate for 10-15 min at 25-30° C. and filtered off sodium sulfate and washed with ethyl acetate (20 ml). The filtrate mother liquors were distilled at 40-50° C. under reduced pressure to get a solid compound. Yield: 35 g (0.7 w/w, based on input).

### Step 4

### Preparation of (R)—N-benzyl-2-(dibenzylamino)-3-methoxypropanomide (N,N-dibenzyl-O-methyl d-serinamide)

N,N-dibenzyl-O-methyl-D-serine (30 g, 0.1052 mol) was dissolved in methylene chloride (250 ml) at 25-30° C. and the resulting solution was cooled to −20° to −15° C. N-methyl morpholine (10.1 g, 0.1105 mol) was added at −20° to −15° C., followed by isobutyl chloroformate (15.09 g, 0.11052 mol) in methylene chloride (50 ml) was added at −20° to −15° C. in 30-35 min. The reaction mass was stirred at −20° to −15° C. for 60-70 min. A solution of benzylamine (11.8 g, 0.1105 mol) in methylene chloride (50 ml) was added at −20° to −15° C. in 30-35 min. The reaction mass was stirred at −20° to −15° C. for 60-70 min. Then the temperature of the reaction mass was raised to 25-30° C. DM water (200 ml) was added and the organic layer was separated and washed with aqueous hydrochloric acid (1N HCl, 100 ml), sodium bicarbonate (8% w/v, 100 ml) and DM water (100 ml). The organic layer was separated and distilled at 35-40° C. under reduced pressure to get brown colour oily mass. Yield: 32 g (1.06 w/w, based on input).

### Step 5

### Preparation of (2R)-2-amino 3-methoxypropanomide (O-methyl D-serinamide)

N,N-dibenzyl-O-methyl-D-serinamide (25 g, 0.0644 mol) was dissolved in methanol (160 ml) at 27-30° C. Palladium hydroxide (10%, 9 g) was added under nitrogen atmosphere. The reaction mass was subjected to parr under hydrogen pressure 5 Kg/Cm² at 25-30° C. for 8 h. The reaction mass was filtered through hyflo and washed the bed with methanol (50 ml). The filtrate mother liquors were concentrated under reduced pressure to get a colourless oily mass. Yield: 13 g (Crude).

### Step 6

### Preparation of Lacosamide

O-methyl-D-serinamide (13 g, 0.0559 mol) was dissolved in methylene chloride (100 ml) at 25-30° C. The resulting solution was cooled to 10-15° C. and pyridine (2 ml), followed by acetic anhydride (6.26 g, 0.06137 mol) was added in 10-15 min at 10-15° C. The temperature of the reaction mass was raised to 25-30° C. for 20-30 min and the mass was stirred for 60-70 min at 25-30° C. DM water (26 ml) was added and the mass was stirred for 10-15 min at 25-30° C. The organic layer was separated and washed with sodium bicarbonate (8%, 26 ml) and DM water (26 ml). The organic layer was separated and concentrated at 35-40° C. under reduced pressure to get a solid compound (13 g). The crude compound was purified optionally by flash chromatography or recrystallized from isopropyl acetate or DM water or mixture thereof.

Chiral purity: ≧99% (by HPLC, by area normalization)

Chromatographic purity: ≧99% (by HPLC, by area normalization)

### EXAMPLE-4

### Step 1

### Preparation of (2R)-2-amino-3-hydroxy-N-(phenylmethyl)propanamide (D-Serinamide)

N-Boc-D-serine (50 g, 0.2436 moles) was suspended in methylene chloride (300 ml) at 25-30° C. Suspension was cooled to 0-5° C. and N-methylmorpholine (25.9 g, 0.2560 moles) was added at 0-5° C. Simultaneously isobutyl chloroformate solution was prepared by adding isobutyl chloroformate (35.0 g, 0.2562 moles) to methylene chloride (125 ml) at 0-15° C. and cooled to −15° to −10° C. Precooled N-Boc-D-serine solution (0-5° C.) was added to isobutyl chloroformate solution at −15° to −10° C. Further, the mass was stirred for 30-40 min at −15° to −10° C. A solution of benzylamine [Prepared by dissolving (27.4 g, 0.2557 moles) in 25 ml of methylene chloride at 20-30° C.] was added to the,reaction mass at −15° to 0° C. Reaction mass was stirred for 60-70 min at −5° to 0° C. DM water (75 ml) was added to the reaction mass and stirred for 10-15 min at −5° to 5° C. and then warmed the reaction mass to 25-30° C. Organic layer was separated and washed with aqueous acetic acid (2×75 ml, 25-30° C.) at 25-30° C. to remove free benzylamine. Organic layer was separated and methylene chloride (260-290 ml) was distilled out partially at 35-40° C. under reduced pressure from reaction mass. Concentrated mass was cooled to 20-25° C. Concentrated hydrochloric acid (Assay, 35% w/w, 63.5 g) was added to the concentrated reaction mass containing Boc-D-serinamide (Step-I Part-A) in 15-25 min at 20-30° C. Reaction mixture was stirred for 60-70 min at 25-30° C. After completion of reaction, DM water (100 ml) was added to the reaction mass and the reaction mass was stirred for. 15-20 min at 25-30° C. Aqueous layer (containing product) was separated and washed with methylene chloride (2×50 ml) at 25-30° C. Sodium chloride (30 g) was added to the aqueous layer at 25-30° C. and stirred for 20-30 min at 25-30° C. The pH of the aqueous solution was adjusted to 11.0-11.5 with ˜50% w/w aqueous sodium hydroxide solution at 25-30° C. Thereafter, the reaction mass was cooled to 20-23° C. and seeded with Lacosamide Stage-I product (0.15 g). The slurry was stirred for 2 h±10 min at 20-23° C. The product was filtered and washed with prechilled methylene chloride (2×30 ml, 0-5° C.). Product was dried at 40-50° C. under reduced pressure (<50 mm Hg).

Yield: 39 g

Chromatographic purity: ≧98%; (By HPLC, by area normalization).

### Step 2

### Preparation of (2R)-2-(acetylamino)-3-hydroxy-N-(phenylmethyl)propanamide (Acetyl-D-serinamid)

D-Serinamide (Step-I product, 30 g, 0.1544 moles) was suspended in methylene chloride (300 ml) at 25-30° C. and the slurry was cooled to 5-7° C. Acetic anhydride (17.35 g, 0.1699 moles) was added to the above slurry uniformly at 5-12° C. Reaction slurry was stirred for 60-70 min at 10-15° C. Thereafter, toluene (300 ml, 20-30° C.) was added to the reaction slurry at 10-15° C. Slurry was cooled to 5-10° C. and stirred for 2 h±10 min. Product was filtered and washed with methylene chloride (40 ml, 0-5° C.) at 15-20° C. Compound was dried at 40-50° C. under reduced pressure (<50 mm Hg).

Yield: 33 g

Chromatographic purity: ≧98.50%; (By HPLC, by area normalization).

### Step 3

### Preparation of (2R)-2-(acetylamino)-3-methoxy-N-(phenylmethyl)propanamide (Lacosamide Crude)

Acetyl-D-serinamide (Step-II, 50 g, 0.2116 moles) was suspended in 5.5% w/w aqueous dimethoxyethane (735 ml) at 25-30° C. Slurry was cooled to 0-3° C., dimethyl sulfate (58.74 g) was added at 0-3° C. in 10-15 min and stirred for 5-10 min at 0-3° C. Aqueous sodium hydroxide solution (20% w/v, 72 ml, 0.36 moles) was added in 2 h±10 min at 0-3° C. Thereafter, reaction mass was stirred at 0-5° C. for 10-12 h. Stirring was stopped and allowed for layer separation for 20-30 min and separated the organic layer. 12% w/v Aqueous ammonia (100 ml) was added to the organic layer at 5-25° C. Reaction mixture was stirred for 1 h±10 min at 20-25° C. Solution of sodium chloride (30% w/v, 50 ml) was added. The mass was stirred for 15-20 min at 25-30° C. The organic layer was separated and dimethoxy ethane was distilled out at 30-40° C. under reduced pressure till no more solvents distilled out. Methylene chloride (500 ml) was added at 25-30° C. and stirred for 10-15 min. DM water (100 ml) was added at 20-25° C. to the organic layer. The reaction mixture was stirred for 20-30 min at 20-25° C., allowed for 20-30 min for layer separation and separated the organic layer. 15% w/v Aqueous sodium chloride solution was added at 20-25° C. Reaction mixture was stirred for 20-30 min at 20-25° C., allowed for 20-30 min for layer separation and separated the organic layer. Mixture of 1,2-dimethoxy-ethane and methylene chloride is distilled out at <40° C. under reduced pressure (<100 mm Hg) till no more solvent distils out.

### Isolation of Lacosamide Crude

Isopropyl acetate (200 ml) was added to the above concentrated mass at 25-30° C. and the slurry was stirred for 1 h±5 min at 25-30° C. Product was filtered and washed with isopropyl acetate (2×50 ml, 25-30° C.). Wet product was dried at 40-50° C. under reduced pressure (<50 mm Hg).

Yield (crude): 30 g

Chromatographic purity: ˜99%; by HPLC, by area normalization.

### Step 4

### Purification of (2R)-2-(acetylamino)-3-methoxy-N-(phenylmethyl)propanamide (Lacosamide Crude)

### Purification

Lacosamide crude (30 g) was dissolved in methylene chloride (300 ml) at 25-30° C. Carbon (0.9 g) was added at 25-30° C. and stirred for 20-30 min at 25-30° C. Solution was filtered through hyflo and the bed was washed with methylene chloride (2×15 ml, 25-30° C.). The filtrate was collected and the methylene chloride was distilled out at 35-40° C. under reduced pressure till no more solvent distills out. Isopropyl acetate (330 ml) has added at 35-40° C. Mixture of methylene chloride and isopropyl acetate (30 ml) is distilled out at 35-40° C. under reduced pressure. Temperature of the suspension was raised to 45-50° C. Slurry was stirred for 4 h±10 min at 45-50° C. Slurry was cooled to 20-25° C. in 60-70 min and stirred for 2 h±10 min at 20-25° C. Product was filtered and washed with isopropyl acetate (2×25 ml, 25-30° C.). Product, was dried at 40-50° C. under reduced pressure (<50 mm Hg) till LOD is achieved <0.5% (Determined on 1 g at 50-60° C. for 1 h under reduced pressure of 20 mm Hg).

Yield: 24.8 g

Chromatographic purity: 99.9%; By HPLC, by area normalization.

Chiral purity: 100%; By HPLC, by area normalization

### EXAMPLE-5

### Purification of Lacosamide

### Method a

Lacosamide (6:5 g) was suspended in DM water (65 ml) at 25-30° C., temperature was raised to 65-70° C. and stirred the mass for 60-70 min at 65-70° C. The solution was cooled to 30-35° C. in 40-50 min. Further, the solution was cooled to 0-5° C. in 30-40 min and the suspension was stirred for 40-50 min at 0-5° C. The compound was filtered and washed with prechilled water (10 ml, 0-5° C.). The compound was dried at 40-50° C. under diminished pressure till to get constant weight. Yield: 3.5 g, Chiral purity: 99.5%, HPLC purity: 99%.

### Method b

Lacosamide (30 g) was suspended in isopropyl acetate (150 ml) at 25-30° C., temperature was raised to 40-45° C. and stirred the suspension for 60-70 min at 40-45° C. The product was filtered and washed with isopropyl acetate (2×45 ml). The compound was dried at 40-50° C. under diminished pressure to constant weight. Yield: 25 g, Chiral purity: 99.97%, HPLC purity: 99.3%.

We claim:

1. A process for the preparation of Lacosamide of Formula I, 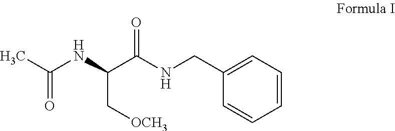 [SMILES: COC[C@@H](NC(C)=O)C(=O)NCc1ccccc1]
comprising O-methylating a compound of Formula (V) in the presence of a methylating agent and a base to produce Lacosamide of Formula (I); 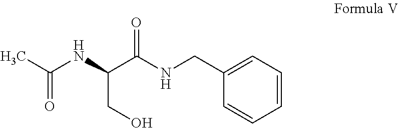 [SMILES: CC(=O)N[C@H](CO)C(=O)NCc1ccccc1]
with proviso that the O-methylation is not carried out in the presence of silver oxide.

2. The process according to claim 1, wherein the methylating agent used in O-methylation step is selected from methyl iodide, methyl chloride, methyl bromide, methyl fluoride, dimethyl sulfate, trimethyl silyldiazomethane, dimethyl sulfoxide (DMSO) or mixtures thereof.

3. The process according to claim 1, wherein the base used in O-methylation step is selected from sodium hydroxide, potassium hydroxide, sodium carbonate, potassium carbonate, sodium bicarbonate, potassium bicarbonate or mixtures thereof.

4. The process according to claim 1, wherein the O-methylation is carried out in the presence of a solvent selected from tetrahydrofuran (THF), dichloromethane (MDC), dimethyl sulfoxide (DMSO), acetonitrile (MeCN), ethyl acetate, acetone, monoglyme, diglyme or mixtures thereof.

5. The process according to claim 1, wherein the O-methylation is optionally carried out in the presence of a phase transfer catalyst (PTC) selected from tetraethylammonium-p-toluenesulfonate, tetrapropylammonium trifluoromethane sulfonate, tetraphenylphosphonium hexafluoroantimonate, acetylpyridinium bromide, triphenylmethyl triphenylphosponium chloride, benzyltriethylammonium chloride, benzyltrimethylammonium chloride, benzyltriphenylphosphonium chloride, benzytributylammonium chloride, butyltriethylammonium bromide, butyltriphenylphosphonium bromide, cetyltrimethyl ammonium bromide, cetyltrimethyl ammonium chloride, ethyltriphenylphosphonium bromide, ethyltriphenylphosphonium iodide, methyltrioctylammonium bromide, methyltriphenylphosphonium bromide, methyltriphenylphosphonium iodide, phenyltrimethylammonium chloride, tetrabutylammonium hydroxide, tetrabutylammonium perchlorate, tetrabutylammonium bromide, tetrabutylammonium hydrogensulphate, tetrabutylammonium iodide, tetrabutylammonium tetrafluoroborate, tetrabutylammonium thiocyanate, tetraethylammonium hydroxide, tetraethylammonium iodide, tetraethylammonium bromide, tetramethylammonium chloride, tetramethylammonium iodide, tetramethylammonium chloride, tetraoctylammonium bromide, tetraphenylphosphonium bromide, tetrapropylammonium hydroxide, tetrapropylammonium bromide and tributylmethylammonium chloride or mixtures thereof.

6. The process according to claim 1, wherein the compound of formula V is prepared by a process, comprising the steps of:
(i) reacting a compound of Formula XIX; 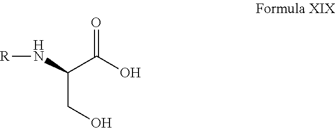
wherein, R represents N-protecting group;
with benzylamine in the presence of a base and an activator of the carboxyl group in a solvent to produce a compound of Formula (XX); 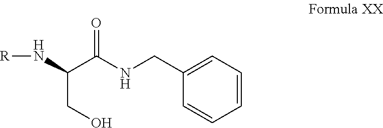
(ii) deprotecting the compound of Formula (XX) in the presence of acid in a solvent to produce a compound of Formula (IV); 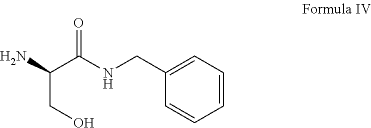 [SMILES: N[C@H](CO)C(=O)NCc1ccccc1]
(iii) acetylating the compound of Formula (IV) in the presence of or absence of a base to produce compound of Formula (V).

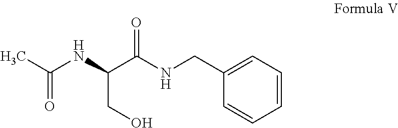 [SMILES: CC(=O)N[C@H](CO)C(=O)NCc1ccccc1]

7. The process according to claim 6, wherein the base used in step (i) is selected from triethylamine, diisopropylethylamine, 1,8-diazabicyclo-[5.4.0]undec-7-ene, 4-methylmorpholine, sodium carbonate, sodium bicarbonate, potassium bicarbonate, calcium carbonate and calcium bicarbonate or mixtures thereof.

8. The process according to claim 6, wherein the activator of the carboxyl group used in step (i) is selected from carbodiimide, isobutyl chloroformate, N,N-carbonyldiimidazole, ethylchloroformate and methylchloroformate or mixtures thereof.

9. The process according to claim 6, wherein the solvent used in step (i) is selected from halogenated solvents such as dichloromethane, ethylene dichloride, and chloroform; ether, toluene, ethyl acetate or mixtures thereof.

10. The process according to claim 6, wherein the acid used in step (ii) is selected from strong acid or mild acid or mixtures thereof.

11. The process according to claim 10, wherein the strong acid is selected from hydrochloric acid, sulphuric acid, trifluoroacetic acid and mixtures thereof.

12. The process according to claim 10, wherein the mild acid is selected from acetic acid, oxalic acid, tartaric, phosphoric acid (H₃PO₄), sodium hydrogen phosphate (Na₂HPO₄) or mixtures thereof.

13. The process according to claim 6, wherein solvent used in step (ii) is selected from aromatic hydrocarbon such as toluene, xylene and aliphatic solvents like chlorinated solvents such as dichloromethane, chloroform, alcohols such as methanol, ethanol, t-butanol, isopropanol, ethyl acetate, cyclopentyl methyl ether or mixtures thereof.

14. The process according to claim 6, wherein the acetylating agent used in step (iii) is selected from acetic anhydride, acetyl chloride, acetic acid or the like or derivatives thereof and the solvent used in acetylation step is selected from chlorinated solvent such as dichloromethane, chloroform; esters such as ethyl acetate, isopropyl acetate or water or mixtures thereof.

15. The process according to claim 6, wherein the solvent used in acetylation step is selected from chlorinated solvent such as dichloromethane, chloroform; esters such as ethyl acetate, isopropyl acetate or water or mixtures thereof.

16. The process according to claim 6, wherein the base used in step (iii) is selected from triethylamine, pyridine, dimethylaminopyridine, N-Methylmorpholine or mixture thereof.

17-41. (canceled)
